(19) United States
(12) Patent Application Publication
(10) Pub. No.: US 2017/0361010 A1
(43) Pub. Date: Dec. 21, 2017

(54) DIALYSIS MACHINE

(71) Applicant: FRESENIUS MEDICAL CARE DEUTSCHLAND GMBH, Bad Homburg (DE)

(72) Inventors: Ralf WOJKE, Hamburg (DE); Paul WIENEKE, Muenster (DE)

(73) Assignee: FRESENIUS MEDICAL CARE DEUTSCHLAND GMBH, Bad Homburg (DE)

(21) Appl. No.: 15/534,380

(22) PCT Filed: Dec. 2, 2015
(86) PCT No.: PCT/EP2015/002429

§ 371 Date: Jun. 8, 2017

(30) Foreign Application Priority Data
Dec. 8, 2014 (DE) 10 2014 018 072.7

(51) Int. Cl. A61M 1/36; A61M 1/16; A61M 1/34

(52) U.S. Cl. CPC A61M 1/361; A61M 1/3403; A61M 1/3644; A61M 1/3413; A61M 1/1603; A61M 2205/3341; A61M 2205/3331; A61M 2205/52; A61M 2205/70; A61M 2206/11; A61M 2230/207; A61M 2205/3334

(57) ABSTRACT

The present invention relates to a dialysis machine having an extracorporeal circuit in which a dialyzer is located which has a chamber on the blood side which is flowed through by blood and a first pressure sensor, which is located upstream of the chamber on the blood side in the direction of flow of the blood, for determining a first pressure value and a second pressure sensor, which is located downstream of the chamber on the blood side in the direction of flow of the blood, for determining a second pressure value, wherein the dialysis machine has first means for determining the pressure difference between the second pressure value and the first pressure value, second means for determining the dynamic viscosity of the blood on the basis of the determined pressure difference, of the blood flow rate through the chamber on the blood side and of one or more characteristic properties of the dialyzer and third means for determining the hematocrit or the hemoglobin value of the blood on the basis of the determined viscosity, and wherein the dialysis machine has a control or regulation unit which is configured such that it sets the blood flow rate and/or the dilution rate and/or the ultrafiltration rate such that the time change of the hematocrit and/or of the hemoglobin value does not exceed a limit value or lies in a desired value range.

The present invention relates to a dialysis machine having an extracorporeal circuit in which a dialyzer is located which has a chamber on the blood side which is flowed through by blood and having a first pressure sensor, which is located upstream of the chamber on the blood side in the direction of flow of the blood, for determining a first pressure value and having a second pressure sensor, which is located downstream of the chamber in the direction of flow of the blood, for determining a second pressure value.

Such dialysis machines are known in different embodiments from the prior art. The blood of the patient is conveyed through the extracorporeal circuit by means of a blood pump. A cleansing or a water elimination takes place in the dialyzer via the dialyzer membrane which usually comprises a plurality of capillary walls. The blood cleansed in this manner arrives back at the patient. While a predominantly diffuse mass transfer takes place via the membrane in hemodialysis, there is also a convective mass transfer in hemodiafiltration caused by a pressure drop over the membrane. Said convective mass transfer is the major value which effects the blood cleansing or the water elimination in hemofiltration.

According to the Hagen-Poiseuille law, in a laminar flow there is a linear relationship between the transported volume of liquid V/t and the pressure difference Δp along the membrane, i.e. the pressure difference between the start and the end of the capillary of a length L, according to the following equation:

V/t=(πr⁴Δp)/(8ηL) (1)

V/t (also called Q) is the blood flow through the capillary; r is the capillary's inner radius and η is the dynamic viscosity of the blood.

Dialyzers typically have a large number n of capillaries (n>10,000) which have a small radius (10 μm<r<110 μm) so that a membrane surface and thus an exchange surface is obtained which is as large as possible. The membrane surface A then results overall as A=2πrLn.

Dialyzers having the same membrane surface A can transport a different fluid volume at the same Δp or can effect a different pressure drop at the same Q since the dialyzers can differ in the parameters r, L and n. With a Newtonian fluid, each individual parameter (n, L, r) already has different effects on the fluid transport in the ideal case, as results from equation (1). The total fluid transport through the dialyzer is proportional to n.

A further factor which can have an influence on the fluid transport is the number and geometry of the pores in the membrane surface or the roughness ε of the membrane surface. If this is taken into account in the above-named equation (1), it could be included in the form of the modified equation (2)

V/t=(πr⁴Δp)/(8ηLε) (2)

from which it can be seen that the flow rate decreases as the roughness increases.

This roughness or the further characteristic properties characterize the dialyzer and can be determined by marking or measurement at the works or can also be determined at the start of a dialysis treatment. Such a determination can take place by pressure measurements upstream and downstream directly at the dialyzer both on the blood side and on the dialyzate side.

With a laminar flow through the capillary, the pressure loss over the length of the capillary is proportional to the mean velocity of the blood in the capillary.

On the transition from a laminar flow to a turbulent flow, the parabolic profile of the flow changes more to a largely uniform velocity over the cross-sectional surface of the capillary. The turbulent fluctuation movement has the consequence of an increased pressure loss Δp over the length of the capillary and an increased wall shear stress. In the turbulent flow, the pressure loss over the capillary length is proportional to the square of the mean velocity.

The dynamic viscosity of the blood changes increasingly due to the liquid elimination over the membrane.

On a huge liquid elimination over the membrane, a huge local increase in the cell density and in the local hematocrit is adopted in the capillary middle. The cells adopt a more and more dense arrangement at the membrane due to the liquid elimination and this results in a hardening of the blood. The blood will move through the hollow fiber at a largely uniform velocity over the cross-section.

With modern capillary dialyzers, the approximation of a laminar pipe flow is no longer generally correct. The capillaries are frequently ondulated; the curves caused by this disturb the laminar flow. There are smaller membrane surfaces and reduced capillary radii which likewise have a negative effect on the laminar flow.

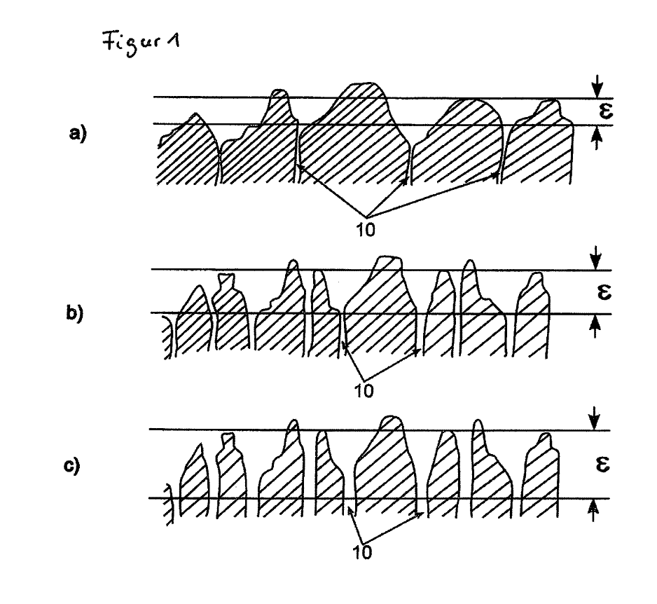

With modern capillaries, more pores are located in the membrane, whereby the liquid permeability through the membrane and also the roughness c of the membrane is increased, as can be seen from FIG. 1 which shows the capillary walls in section and which shows the roughness values for a low-flux membrane a), for a high-flux membrane b) and for a high cut-off membrane c). The inner capillary side is respectively located at the top. The pores are marked by the reference numeral 10.

In modern dialysis processes such as the hemodiafiltration process, a huge increase in the hematocrit up to 70 to 80% can occur which at least locally produces the above-named hardening of the blood.

Starting from the inlet region of the capillary having a physiological hematocrit and a laminar blood flow, a hematocrit non-physiologically increased due to ultrafiltration and a no longer homogeneous flow result in a transition region. Cell aggregates are formed; the parabolic velocity profile flattens out. In the end region, the hematocrit is extremely non-physiological in the total blood volume; the blood hardens and the velocity profile becomes plug-shaped. The local pressure drop increases greatly and the boundary layer between the blood and the membrane dissolves. Substantial interactions arise between cells and the membrane and cell damage may occur.

The cells impact one another within the hollow fiber due to liquid removal and thickening, i.e. the cells can no longer move freely, but rather act on one another. With larger capillary diameters, this can result in cell deformations; with smaller capillary diameters, a regrouping and deformation of the cells can take place.

In the near region of the capillary wall, blood cells are moved along the capillary wall at a medium blood speed and under certain circumstances undergo turbulent fluctuation movements, increased wall shear stress and shear forces as well as deforming and abrasive effects due to the contact with the rough capillary wall.

The cellular components are thickened and pushed onto the membrane with the ultrafiltration of liquid components of the blood over the capillary membrane. Depending on the mechanical consistency and on the design of the capillary membrane, deforming and abrasive effects at the cellular membranes are also conceivable, in particular in the region of the pores.

The interaction of fast flowing blood and of the capillary membrane can also influence the capillary membrane itself under certain circumstances. It is thus conceivable, for example, that the pores are changed, which can in turn have an influence on the blood or on its components.

It can be stated in summary that the above-named effects may under certain circumstances result in an impairment of the integrity of the blood cells: depending on the cell type, this impairment can result in a deformation, in a reduction of the life of the cells, up to a destruction of the cells (hemolysis), or also in the release of cellular components, possibly with systemic consequences (cytokines).

If the blood flow rate is increased, starting from low values, a departure from the region of the laminar blood flow may occur, i.e. the substantially parabolic velocity profile is left behind and a mean velocity profile is generated which is uniform over the cross-sectional area. The transition point depends on the viscosity and on the geometry of the hollow fiber. This transition, which is accompanied by a hardening of the blood, is smooth.

The ultrafiltration results in a liquid elimination over the membrane, i.e. is in principle effective over the total capillary length over the hollow fiber or capillaries. The viscosity of the blood is changed by the liquid elimination. If the ultrafiltration is greatly increased, the character of the blood can change from Newtonian to non-Newtonian. The flow behavior of the blood changes in that the velocity profile is flattened out in the capillary center, i.e. the parabolic profile with the greatest speed in the capillary center and the lowest speed at the capillary wall flattens out. The flow is, however, not turbulent, but rather stiffened.

It results in summary that a high blood flow rate and a high ultrafiltration rate can increase the probability of the occurrence of cell damage of the blood.

The fragility of the blood cells is, in a first approximation, dependent on genetic factors, on the frequency and intensity of the renal replacement therapy, on the residual renal function and on other system diseases and differs from patient to patient. The transition from Newtonian to non-Newtonian behavior depends inter alia on the proportion of cellular components (hematocrit).

It is the underlying object of the present invention to further develop a dialysis machine of the initially named kind such that the probability of the occurrence of such cell damage due to the thickening of the blood is reduced.

This object is satisfied by a dialysis machine having the features of claim 1.

Provision is accordingly made that the dialysis machine has first means for determining the pressure difference between the second pressure value and the first pressure value; that the dialysis machine has second means for determining the dynamic viscosity of the blood on the basis of the determined pressure difference, of the blood flow rate through the chamber on the blood side and of one or more characteristic properties of the dialyzer; that the dialysis machine has third means for determining the hematocrit or the hemoglobin value of the blood on the basis of the determined viscosity; and that the dialysis machine has a control or regulation unit which is configured such that it sets the blood flow rate and/or the dilution rate and/or the ultrafiltration rate such that the time change of the hematocrit and/or of the hemoglobin value does not exceed a limit value or lies within a desired value range.

In accordance with the current invention, the pressure difference on the blood side is determined (preferably directly) before and after the dialyzer and the dynamic viscosity of the blood is determined based on this. In accordance with equation (2), further parameters which enter into the determination of the viscosity are the blood flow (V/t) as well as properties of the dialyzer, in particular its laminar flow resistance which can e.g. be determined within the framework of the dialyzer production.

The hematocrit and/or the hemoglobin value can then be determined from the determined viscosity.

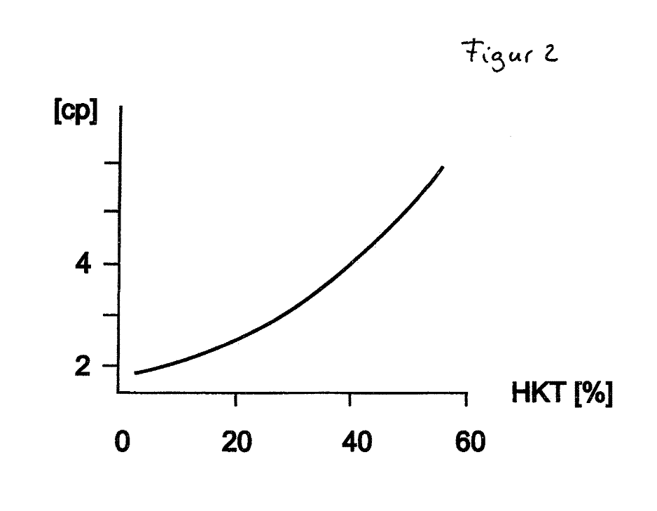

FIG. 2 shows an exemplary relationship between the hematocrit HKT and the dynamic viscosity η.

The relationship shown in FIG. 2 applies to an idealized patient and can be used for all patients with the lack of precision associated therewith. It is, however, advantageous to use a relationship individual to the patient in order thus to obtain a result for the hematocrit and/or the hemoglobin value which is as exact as possible.

The control or regulation unit of the dialysis machine compares the time increase in the hematocrit and/or in the hemoglobin value and compares this with an upper limit value or checks whether it lies in a desired value range.

If this is the case, no intervention by the dialysis machine is required since a specific increase during the treatment is normal.

If, however, an exceeding of the limit value occurs or if there is a departure from the desired value range, the dialysis machine changes the blood flow rate at the instigation of the control or regulation unit by a change in the speed of the blood pump and/or changes the substitution rate via the change in the speed of a substitution pump at which a substitution fluid is supplied to the extracorporeal circuit. Alternatively or additionally, the ultrafiltration rate is changed.

Provision is, however, preferably made that the ultrafiltration rate is not used for setting the time change of the hematocrit and/or of the hemoglobin value since it should adopt a specific value or a specific profile to achieve the desired dry weight of the patient.

The limit value or the desired value range can be fixedly preset or can be determined experimentally such as by an increase of the cell debris or free hemoglobin with a simulated dialysis treatment in the blood laboratory.

The properties of the dialyzer which flow into the determination of the viscosity can comprise the flow resistance of the dialyzer which results with a laminar flow. Said flow resistance can, for example, be determined after the manufacture of the dialyzer at the works or before the treatment by means of a flushing fluid or also at the start of the treatment by means of the blood itself.

It is furthermore conceivable that the dialyzer chamber on the blood side comprises a plurality of capillaries and that the properties of the dialyzer comprise the length of the capillaries and/or their inner dimensions and/or their number and/or their shape and/or their wall structure and/or the roughness of the capillary walls.

The properties of the dialyzer, in particular its flow resistance, can be determined by measuring the pressure drop over the capillary length Δp with specific flows and media from equation (1) or (2).

If the properties of the dialyzer are known, the dynamic viscosity η of the blood can be determined with a known or measured blood flow (V/t or Q) by the measurement of the pressure loss over the capillary length.

Provision is made in a further embodiment of the invention that the third means have a memory in which a relationship between the hematocrit or hemoglobin value of the blood and the viscosity is stored and that the third means are configured such that the hematocrit or the hemoglobin value of the blood is determined on the basis of this relationship.

As shown in FIG. 2, there is a non-linear relationship between the two values.

It is conceivable that the relationship is fixedly preset. In this case, a fixed relationship between the hematocrit and the viscosity is used independently of the patient.

If the blood flow resistance is determined in specific patients, e.g. at the start of the treatment, a correction factor can be calculated by a comparison with the hematocrit or hemoglobin known from the laboratory which improves the precision of these blood viscosity/hematocrit curves on an individual patient basis and which thus improves the precision of the determined hematocrit and/or of the hemoglobin value.

The third means can be configured such that the hematocrit or the hemoglobin value is determined by interpolation or by extrapolation. Other types of calculation are also conceivable and covered by the invention.

If the dialyzer is determined sufficiently precisely, i.e. if its characteristic properties are determined, a conclusion can be drawn on the viscosity from the measured pressure values at the dialyzer and from said viscosity on the hematocrit and/or on the hemoglobin value. If, in addition, an individual patient calibration is carried out, influences of the blood and individual, possibly pathological, influences on the blood viscosity, can be compensated or taken into account.

If the dialyzer is determined sufficiently exactly, i.e. if its characteristic properties have been determined, a conclusion can be drawn on the viscosity from an increase in the measured pressure drop over the dialyzer length and from said viscosity on the hematocrit and/or on the hemoglobin value or their time change.

The first to third means are preferably anyway components of a dialysis machine so that no special additional disposable, etc. is necessary, whereby costs can be saved.

It is conceivable that the characteristic properties of the dialyzer are obtained by measurement with blood or by measurement with another fluid, in particular by measurement with a flushing fluid. A “calibration”, i.e. the determination of the flow resistance on the blood side, can thus be carried out during or after the priming. It is also possible to carry out such a determination ex works. If the viscosity of the liquid is known or if it can be eliminated from the equation system by a plurality of measurements and if the pressure loss is measured over the length of the capillaries, the characteristic properties and in particular the flow resistance of the dialyzer can be determined therefrom.

This calibration can e.g. also be carried out by multi-point measurements at different flow rates and ultrafiltration rates.

The dialysis machine can have a chamber flowed through by dialysis solution on the dialyzate side and a third pressure sensor, which is located upstream of this chamber in the direction of flow of the dialysis solution, for determining a third pressure value and a fourth pressure sensor, located downstream of this chamber in the direction of flow of the dialysis solution, for determining a fourth pressure value.

The dialysis machine can be a hemodialysis machine, a hemofiltration device or a hemodiafiltration device. Accordingly, the term “dialyzer” can also be understood as a hemodialyzer or also a hemofilter which is used in hemodiafiltration or in hemofiltration.

The dialysis machine preferably has a predilution line which opens into the extracorporeal circuit upstream of the dialyzer in the direction of flow of the dialyzer. The dilution rate in this case is preferably the infusion rate of the substitution fluid supplied through the predilution line. A postdilution is also possible and covered by the invention.

Further details and advantages of the invention will be explained in more detail with reference to an embodiment shown in the drawing. There are shown:

FIG. 1: different schematic longitudinal sectional views through different membrane types; and

FIG. 2: a schematic representation of the dependence of the blood viscosity on the hematocrit.

The flow resistance on the blood side is measured before the start of the dialysis treatment via the pressure sensors present in the hemodialysis device, i.e. the “calibration” does not take place in the works. This measurement can be carried out automatically during, or also after the priming of the dialysis machine.

During priming, the measurement takes place e.g. by means of a flushing solution such as a physiological saline solution. After the priming, the measurement takes place by means of the patient's blood.

There is the advantage in the first case that the patient does not have to be present; however, there is the disadvantage that only the flow resistance is measured using the saline solution, which only represents an approximation for the blood resistance on being flowed through by blood.

Alternatively to this procedure, the “calibration” can take place at the production site of the dialyzer and a transfer of the result to the dialysis machine can take place.

The geometry of the dialyzer such as the number of capillaries, their lengths, their inner capillary radius, the design of the membrane (such as the number, the size and the geometry of the pores) and optionally the properties of the materials used (such as the elasticity, hardness, compliance) inter alia enter into the resistance on the blood side.

It is conceivable that the first and last dialyzers are measured after the production of a lot of dialyzers and the dialyzers of the total lot are provided with the value or mean value of the determined flow resistance (e.g. on an RFID chip, barcode or in another suitable data store). This value can be read in by the dialysis machine for the treatment, preferable before the treatment.

If the dialyzer has been calibrated in this manner, i.e. if its properties and in particular its flow resistance are known, the dynamic viscosity can be determined by the measurement of the pressure loss over the length of the dialyzer.

The hematocrit and/or the hemoglobin value can be determined from the viscosity on this basis. Provision can be made to improve the precision of this determination that the blood flow resistance is measured for the individual patient at the start of the treatment and a correction factor is calculated by a comparison with the hematocrit and/or the hemoglobin value known from the laboratory which improves the precision of the blood viscosity and hematocrit curves shown by way of example in FIG. 2 on an individual patient basis.

The determination of the hematocrit and/or the hemoglobin value can take place using means of the dialysis machine, i.e. no additional elements separately provided therefor are preferably present.

The concentration of the blood, i.e. the increase in the hematocrit ΔHKT over time takes place constantly in a first approximation with unchanged treatment parameters during the dialysis treatment.

If the measured hematocrit increases unexpectedly and abruptly or more steeply in a smooth transition, that is if ΔHKT/Δt exceeds a limit value, this can be interpreted as an increase in the viscosity due to the non-Newtonian effects at the capillary surface. If it is found during the treatment that the time increase exceeds a limit value, the blood flow rate and/or the dilution rate, preferably the predilution rate, is varied by means of a control or regulation unit of the dialysis machine such that the expected ΔHKT/Δt is again adopted or lies in a specific desired value range which can e.g. be present as a window around a desired value. The blood flow rate and/or the ultrafiltration rate is/are preferably reduced on a falling below of the limit value and/or the dilution rate is increased.

A regulation of the ΔHKT/Δt preferably takes place, with the blood flow rate and/or the dilution rate and/or the ultrafiltration rate being able to serve as control variables.

A change of the ultrafiltration range for setting the ΔHKT/Δt is admittedly generally conceivable and covered by the invention. It is, however, preferred if it remains constant or follows a predefined profile to be able to achieve the desired weight loss at the end of the treatment.

1. A dialysis machine having an extracorporeal circuit in which a dialyzer is located which has a chamber on the blood side which is flowed through by blood and a first pressure sensor, which is located upstream of the chamber on the blood side in the direction of flow of the blood, for determining a first pressure value and a second pressure sensor, which is located downstream of the chamber on the blood side in the direction of flow of the blood, for determining a second pressure value,
characterized in that
the dialysis machine has first means for determining the pressure difference between the second pressure value and the first pressure value;
the dialysis machine has second means for determining the dynamic viscosity of the blood on the basis of the determined pressure difference, of the blood flow rate through the chamber on the blood side and of one or more characteristic properties of the dialyzer;
the dialysis machine has third means for determining the hematocrit or the hemoglobin value of the blood on the basis of the determined viscosity; and
the dialysis machine has a control or regulation unit which is configured such that it sets the blood flow rate and/or the dilution rate and/or the ultrafiltration rate such that the time change of the hematocrit and/or of the hemoglobin value does not exceed a limit value or lies within a desired value range.

2. A dialysis machine in accordance with claim 1, characterized in that the control or regulation unit is configured such that the ultrafiltration rate is not used to set the change of the hematocrit and/or of the hemoglobin value.

3. A dialysis machine in accordance with claim 1, characterized in that the limit value or the desired value range is fixedly predefined or is determined experimentally.

4. A dialysis machine in accordance with claim 1, characterized in that the properties of the dialyzer comprise the flow resistance of the dialyzer which results with a laminar flow.

5. A dialysis machine in accordance with claim 1, characterized in that the dialyzer chamber on the blood side comprises the lumen of a plurality of capillaries; and in that the properties of the dialyzer comprise the length of the capillaries and/or their inner dimensions and/or their number and/or their shape and/or their wall structure and/or the roughness of the capillary wall.

6. A dialysis machine in accordance with claim 1, characterized in that the third means have a memory in which a relationship between the hematocrit or hemoglobin value of the blood and the viscosity is stored and that the third means are configured such that the hematocrit or the hemoglobin value of the blood is determined on the basis of this relationship.

7. A dialysis machine in accordance with claim 6, characterized in that the relationship is fixedly preset.

8. A dialysis machine in accordance with claim 6, characterized in that the relationship is determined by means of a correction factor on an individual patient basis.

9. A dialysis machine in accordance with claim 6, characterized in that the third means are configured such that the hematocrit or hemoglobin value is determined by interpolation or by extrapolation.

10. A dialysis machine in accordance with claim 1, characterized in that the dialysis machine has means for reading in the characteristic properties of the dialyzer which are in communication with the second means for determining the viscosity.

11. A dialysis machine in accordance with claim 1, characterized in that the first to third means are components of a dialysis machine anyway present so that no additional elements are required to carry out the measures carried out by the first to third means.

12. A dialysis machine in accordance with claim 1, characterized in that the characteristic properties of the dialyzer are obtained by measurement with blood or by measurement with another fluid, in particular by measurement with a flushing liquid.

13. A dialysis machine in accordance with claim 1, characterized in that the dialysis machine has a chamber flowed through by dialysis solution on the dialyzate side and a third pressure sensor, which is located upstream of the chamber at the dialyzate side in the direction of flow of the dialysis solution, for determining a third pressure value and a fourth pressure sensor, located downstream of the chamber at the dialyzate side in the direction of flow of the dialysis solution, for determining a fourth pressure value.

14. A dialysis machine in accordance with claim 1, characterized in that the dialyzer is a hemodialyzer or a hemofilter.

15. A dialysis machine in accordance with claim 1, characterized in that the dialysis machine has a predilution line which opens into the extracorporeal circuit upstream of the dialyzer in the direction of flow of the dialyzer; and in that the dilution rate is the infusion rate of a substitution fluid supplied through the predilution line.
